(19) United States
(12) Patent Application Publication
(10) Pub. No.: US 2018/0151254 A1
(43) Pub. Date: May 31, 2018

(54) HIGH-SPEED SIMILAR CASE SEARCH METHOD AND DEVICE THROUGH REDUCTION OF LARGE SCALE MULTI-DIMENSIONAL TIME SERIES HEALTH DATA TO MULTIPLE DIMENSIONS

(71) Applicant: Electronics And Telecommunications Research Institute, Daejeon (KR)

(72) Inventors: Youngwoong HAN, Daejeon (KR); Ho-Youl JUNG, Daejeon (KR); Jae Hun CHOI, Daejeon (KR); Dae Hee KIM, Daejeon (KR); Minho KIM, Daejeon (KR); Seunghwan KIM, Daejeon (KR); YoungWon KIM, Daejeon (KR); Donghun LEE, Daejeon (KR); Myung-Eun LIM, Daejeon (KR)

(21) Appl. No.: 15/828,180

(22) Filed: Nov. 30, 2017

(30) Foreign Application Priority Data
Nov. 30, 2016 (KR) 10-2016-0161990
Nov. 10, 2017 (KR) 10-2017-0149877

(51) Int. Cl. G16H 10/60; G16H 50/20; G06F 15/18; G06F 17/30; G06K 9/62

(52) U.S. Cl. CPC G16H 10/60; G16H 50/20; G06F 15/18; G06F 2216/03; G06K 9/6232; G06F 17/30592; G06F 17/30539

(57) ABSTRACT

Provided are a search method and device for searching for a case similar to user's health data at high-speed from large scale multi-dimensional time series health data. The method includes preprocessing health data inputted through an interface circuit, performing a multi-dimensional feature extraction learning based on machine learning on the preprocessed health data, and generating one or more feature extraction models for dimension reduction based on the multi-dimensional feature extraction learning.

### CROSS-REFERENCE TO RELATED APPLICATIONS

This U.S. non-provisional patent application claims priority under 35 U.S.C. § 119 of Korean Patent Application No. 10-2016-0161990, filed on Nov. 30, 2016, and Korean Patent Application No. 10-2017-0149877, filed on Nov. 10, 2017, the entire contents of which are hereby incorporated by reference.

### BACKGROUND

The present disclosure relates to a search method and device for searching for a case similar to user's health data at high-speed from large scale multi-dimensional time series health data.

With the recent economic development and rising income levels, modern society is becoming an aging society gradually, and the prevalence of various diseases such as chronic diseases due to changes in lifestyle and wrong eating habits is increasing, so that people's interest in health and well-being is increasing.

In addition, due to the development of industrial technologies and information and communication technologies, the era of big data, in which a large amount of information and data may not be measured, is coming.

In line with such a social change, big data in the medical field may be utilized as a tool to solve the desire for improvement of quality of life according to social changes, so that social interest in big data is increasing.

Accordingly, in recent years, health big data-based service is starting by collecting public health big data provided from many people or from major domestic medical institutions or government, searching for a case identical or similar to that of a particular user based on the personal health data of the particular user (e.g., a patient), and predicting the future health trend of the user based on the search results to use it as a reference material for proper care and health promotion.

For example, services such as Patient Like Me collect a large number of people's health data and provide a search service to search for health data (symptoms and prescriptions) of people who suffer the same disease as a particular user, and based on the results of the search, provide a reference material for promoting the health of specific users. In such a way, health big data based services may search for similar cases of people who show health conditions similar to that of the user and predict future health states of the user with reference to their health changes, and based on the symptoms, lifestyle, eating habits, prescription, etc. obtained from the similar cases, provide a personal health promotion method suitable for each user.

As described above, since the result of the similar case search based on the user's personal health data is reference information that may be utilized as a reference material for the user's health prediction or health promotion improvement, in order to provide smooth health services, a similar case search close to real-time is required.

However, since health data is a record of health values for each health feature (e.g., blood sugar, cholesterol, preference food, family history, etc.) over time of treatment obtained after people have regular health examinations, the health data has large scale multi-dimensional time series characteristics.

In order to calculate the similarity between health data with characteristics of such large scale multi-dimensional time series, various health number information according to the time series should be compared with each other. Therefore, the time complexity is very high and the time spent in searching for similar cases takes too long.

### SUMMARY

The present disclosure provides a device and method for applying a machine learning based feature extraction technique, which reduces specific data dimension, to health data with characteristics of large scale multi-dimensional time series to reduce the dimension of the health data to multi-dimensions, and grouping and partitioning a plurality of health data reduced in multi-dimensions into health data with high similarity, thereby enabling similar case searches close to real-time to provide health promotion services to the user based on user's personal health data.

An embodiment of the inventive concept provides a method performed by a device including one or more processors for similar case search on multi-dimensional health data. The method includes: preprocessing health data inputted through an interface circuit; performing a multi-dimensional feature extraction learning based on machine learning on the preprocessed health data; and generating one or more feature extraction models for dimension reduction based on the multi-dimensional feature extraction learning.

In an embodiment, the method may further include: reducing a dimension for a feature of health data by applying the preprocessed health data to the generated one or more feature extraction models; extracting the feature of the reduced dimension; and grouping the health data of the reduced dimension by each partition based on the extracted feature.

In am embodiment, the method may further include: when personal health data of a user for a similar case search is inputted as query data through the interface circuit, preprocessing the query data; reducing the dimension of the feature for the personal health data of the user by applying the preprocessed query data to the generated one or more feature extraction models; and extracting the query data of the reduced dimension.

In an embodiment, the method may further include: matching the query data of the reduced dimension to health data of a grouped partition; calculating a similarity between the health data of the matched partition and the query data; and outputting health data having the similarity that is greater than or equal to a set value.

In an embodiment, the calculating of the similarity may include: when the number of the health data of the matched partition is less than a critical value, matching health data of a partition adjacent to the matched partition to the query data of the reduced dimension; and calculating the similarity between the health data of the adjacent partition and the query data.

In an embodiment, the one or more feature extraction models may be generated by applying at least one of a Principal Component Analysis (PCA) technique, a Deep Network Learning technique, and a Singular Value Decomposition (SVD) technique.

In an embodiment of the inventive concept, a device configured to provide a similar case search on multi-dimensional health data includes: an input/output interface configured to receive health data; and a controller configured to preprocess the received health data and perform a multi-dimensional feature extraction learning based on machine learning on the preprocessed health data to generate one or more feature extraction models for dimension reduction.

In an embodiment, the controller may be configured to reduce a dimension for a feature of health data by applying the preprocessed health data to the generated one or more feature extraction models, extract the feature of the reduced dimension, and group the health data of the reduced dimension by each partition based on the extracted feature.

In an embodiment, when personal health data of a user for a similar case search is inputted as query data through the interface circuit, the controller may be further configured to preprocess the query data, reduce the dimension of the feature for the personal health data of the user by applying the preprocessed query data to the generated one or more feature extraction models, and extract the query data of the reduced dimension.

In an embodiment, the controller may be further configured to match the query data of the reduced dimension to health data of a grouped partition, calculate a similarity between the health data of the matched partition and the query data; and output health data having the similarity that is greater than or equal to a set value.

In an embodiment, in order to output the health data having the similarity that is greater than or equal to the set value, the controller may be further configured to, when the number of the health data of the matched partition is less than a critical value, match health data of a partition adjacent to the matched partition to the query data of the reduced dimension; and calculate the similarity between the health data of the adjacent partition and the query data.

In an embodiment, the one or more feature extraction models may be generated by applying at least one of a Principal Component Analysis (PCA) technique, a Deep Network Learning technique, and a Singular Value Decomposition (SVD) technique.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a conceptual diagram for schematically explaining a high-speed similar case search method and a device thereof by reducing a large scale multi-dimensional time series health data to multi-dimensions according to an embodiment;

FIG. 2 is a block diagram illustrating a configuration of a high-speed similar case search device according to an embodiment;

FIG. 3 is a workflow illustrating a procedure for searching for similar cases at high speed using personal health data of a user according to an embodiment;

FIG. 4 is a diagram illustrating a process of partitioning large scale multi-dimensional time series health data according to an embodiment; and

FIG. 5 is a diagram illustrating a similar case search process according to an embodiment.

### DETAILED DESCRIPTION

According to an embodiment, in order to provide health information to a user, a health information database that contains health information including treatment labels for a plurality of patients and health information not including treatment labels may be utilized. For example, a device may group similar health information through a Euclid distance similarity calculation method, and provides to a specific patient the treatment label information in the grouped health information similar to the health information of the specific patient. However, to group health data with similar features, since a device matches all the information of each label of health information to calculate similarity and groups health information, the time required for grouping is very long and computational complexity is very high. In addition, in order to provide treatment label information of people similar to a particular patient by utilizing grouped data, since the similarity calculation is performed by matching the whole health information of the specific patient and the grouped data one by one, it may take a long time to get results.

According to an embodiment, health consulting information that considers body information similarity may be provided to a user. For example, a device may provide accurate health consulting information that is mapped to the user's body information by searching for health consulting information of a person having body information similar to the user's body information. In such an embodiment, a device may search for a person's body information similar to the user's body information and provide the user's health consulting information based on the consulting information of the corresponding person. However, since the health information is searched by comparing the body information of the user and the body information of a plurality of others one by one, the time complexity of the operation performed to provide the health information is high. In addition, since the search based body information includes the health features measured over time, it may have large scale multi-dimensional time series characteristics. When similarity calculations are performed on these data, it takes a long time and also computational complexity is very high.

Hereinafter, preferred embodiments of the inventive concept will be described in detail with reference to the accompanying drawings. Like reference numerals in each drawing represent like elements.

Hereinafter, the term “unit” or “module” used in the specification may mean a hardware component or an electronic circuit such as Field Programmable Gate Array (FPGA) or Application Specific Integrated Circuit (ASIC).

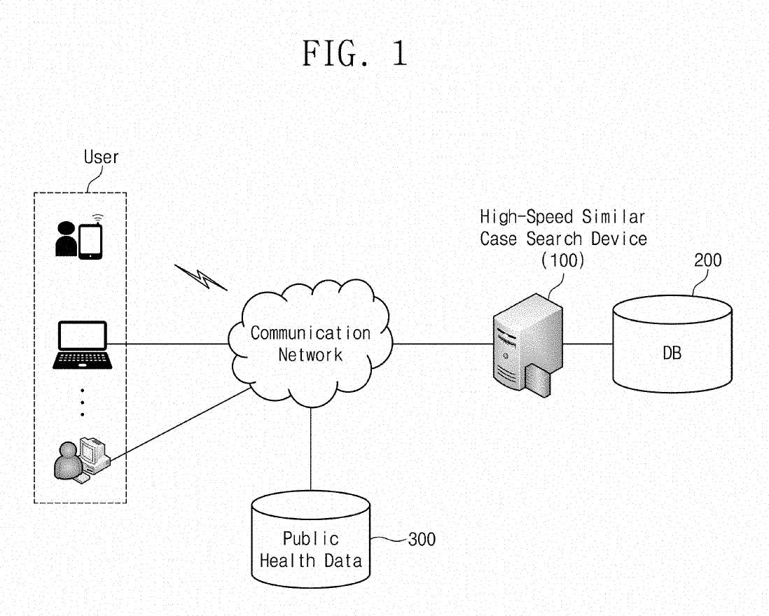

FIG. 1 is a conceptual diagram for schematically explaining a high-speed similar case search method and a device thereof by reducing a large scale multi-dimensional time series health data to multi-dimensions according to an embodiment.

As shown in FIG. 1, a high-speed similar case search device 100 establishes a database 200 for similar case search to provide a similar case search service to a user via a wire/wireless communication network.

The high-speed similar case search device 100 may be provided in a service organization for personal health promotion such as a hospital, a clinic or the like, or may be implemented in the form of a cloud server or an integrated platform in a wire/wireless network.

The user of FIG. 1 may include a service provider or individual that provides personal health promotion services, such as a medical practitioner who treats a patient in a hospital, a well-being service, a fitness service, etc. Also, a user may access the high-speed similar case search device 100 through a user terminal and search for similar cases at high speed based on the personal health data which is the target of the similar case search.

Also, the high-speed similar case search device 100 may periodically receive public health data from a public health database 300 to establish a database 200 for high-speed similar case search. Here, the public health data may be data that does not include personal information (e.g., resident registration number, telephone number, address, etc.). For example, when the inputted public health data includes personal information, the high-speed similar case search device 100 may delete personal information by itself.

In addition, the public health data may also be provided by large hospitals, government agencies, or users. That is, a government agency or user may be a provider of public health data.

The inputted public health data may be big data having characteristics of a large scale multi-dimensional time series. To reduce the computational complexity of the search time and similarity calculations, the high-speed similar case search device 100 may reduce the dimension of the public health data through a multi-dimension reduction technique, and support the similar case search speed close to real-time.

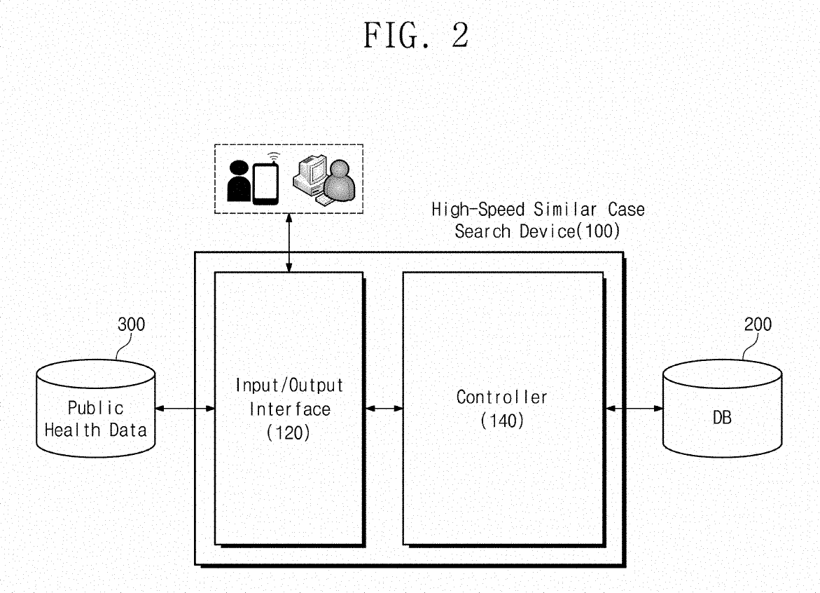

Meanwhile, the multi-dimension reduction technique may generate a feature extraction model for reducing the dimension of the health data by learning the health data, which will be described in detail with reference to FIG. 2.

The high-speed similar case search device 100 may periodically receive the public health data and update the database 200 for the similar case search, thereby keeping the database 200 up-to-date.

Also, the high-speed similar case search device 100 provides a user interface for a user's connection, such as login, input of personal health data, and the like, and also may provide information on the trend of the health state of the user in addition to similar cases of personal health data provided from the user at the user's request.

FIG. 2 is a block diagram illustrating a configuration of a high-speed similar case search device according to an embodiment.

The high-speed similar case search device 100 may include an input/output interface 120 and a controller 140.

The high-speed similar case search device 100 may receive query data including personal health data from a user or receive public health data from a health data provider through the input/output interface 120. The input/output interface 120 may refer to a hardware component or an electronic circuit for exchanging data with an external system or device of the high-speed similar case search device 100.

The input/output interface 120 may include a user interface that allows a user to access and interact with the high-speed similar case search device 100. The user interface may include, for example, a keypad for providing data and communication inputs, a touch pad, a soft key, a keyboard, a microphone, an infrared sensor for receiving a remote signal, or a combination thereof. The input/output interface 120 may include a communication circuit for communicating with an external system or device of the high-speed similar case search device 100. For example, the input/output interface 120 may include a communication circuit enabling wireless communication, wired communication, optical, ultrasonic, or a combination thereof. For example, the input/output interface 120 may include a communication circuit for receiving public health data. The input/output interface 120 may include electronic circuits for interaction.

The controller 140 may search for similar cases based on the user's query data. For example, the controller 140 may generate a feature extraction model for searching for similar cases based on the public health data inputted by the input/output interface 120. The controller 140 according to an embodiment may be an ASIC, an embedded processor, a microprocessor, hardware control logic, a hardware finite state machine (FSM), a digital signal processor (DSP), or a combination thereof. In an embodiment, the controller 140 may include one or more processors or processor cores (not shown).

The controller 140 may preprocess the inputted personal health data and public health data and may generate one or more feature extraction models by learning the inputted public health data (e.g., preprocessed public health data). The controller 140 may extract the features of the inputted public health data using the generated one or more feature extraction models and partition the public health data of the extracted features. The controller 140 may search for similar cases based on the inputted personal health data.

Specifically, the input/output interface 120 may provide a login for a user connected to the high-speed similar case search device 100 based on previously stored user information such as a user ID and a user password. The input/output interface 120 may receive query data from a user who performs the login. The query data may include user's personal health data. That is, the user may input query data including the user's personal health data to the high-speed similar case search device 100 through the input/output interface 120 to search for similar cases similar to the health state of the user. The user does not need to input all of the health features of his personal health data and may select specific health features and input them through the input/output interface 120 and search for similar cases based on the input.

The high-speed similar case search device 100 according to an embodiment accesses the public health database 300 provided by a provider providing public health data through the input/output interface 120 and receives public health data. For example, the high-speed similar case search device 100 may periodically collect public health data based on a period preset by an administrator of the high-speed similar case search device 100.

Also, the input/output interface 120 periodically receives the public health data from a provider providing the public health data connected to the Internet, thereby allowing the database 200 for similar case search to be updated to the latest state. Thus, the user may search for similar cases with the latest data.

The controller 140 may perform a preprocessing process on public health data to effectively and efficiently generate one or more feature extraction models. The preprocessing process may performed by converting the numerical values of health features (e.g., blood glucose, systolic blood pressure, diastolic blood pressure, cholesterol, family history, lifestyle, etc.) into a probability value form between 0 and 1.

In addition, when the controller 140 performs a preprocessing process, if a numerical value is not listed in a specific health feature among a plurality of time-series health features, the controller 140 may average the measured values before and after the corresponding health feature, or substitute it with an intermediate value and insert the substituted value. Moreover, the controller 140 may insert a value of 0 or 1 instead in relation to a health feature that is not represented by a numerical value, such as a preference food, lifestyle (e.g., drinking or smoking), and the like.

Further, based on user's personal health data, a numerical value of the health features of the personal health data included in the query data inputted from the user to search for similar cases in the health data may be converted into a value between 0 and 1 through the preprocessing.

The controller 140 generates one or more feature extraction models to reduce the dimensions of the public health data by applying a machine learning technique to extract features and reduces the dimension of the public health data through the one or more feature extraction models.

That is, if the query data processed by the preprocessing process and the health data are N-dimensions (i.e., the number of the features or the number of the numerical value), the dimension of the public health data may be reduced to k-dimensions (N>k) through the generated feature extraction model.

For example, at least one feature extraction model may extract health features over time from the public health data to reduce the entire public health data having multi-dimensional characteristics to two dimensions (feature1 and feature2), three dimensions (feature1, feature2, and feature3), or more.

The controller 140 may generate one or more feature extraction models by learning the public health data through a machine learning technique for extracting features from specific data, such as PCA techniques, deep network learning techniques, SVD techniques, and so on.

In addition, the controller 140 may generate one or more feature extraction models that may reduce the public health data to different dimensions and store the generated one or more feature extraction models in the database 200.

The feature extraction model may reduce the dimensions of public health data and personal health data processed by the preprocessing process. One or more feature extraction models may be generated to reduce multi-dimensional public health data and personal health data into multi-dimensions of health features over time.

Furthermore, the controller 140 may apply the public health data to one or more feature extraction models to reduce the dimensions of the public health data. That is, the controller 140 may reduce the dimension of the public health data according to the reduction dimension of each feature extraction model in relation to a plurality of feature extraction models.

In addition, the controller 140 performs partitioning on each public health data according to the reduced dimension based on a plurality of public health data of a reduced dimension to group a plurality of health data showing the extracted health feature of similar patterns into similar groups. That is, each similar group including a plurality of grouped health data may be one partition. As a result of the partitioning, the plurality of public health data may be stored by each partition.

That is, the controller 140 may partition a plurality of public health data for each public health data showing health features of similar patterns in each public health data extracted through one or more feature extraction models, and store the health data for each partition. The partitioning may be performed according to the dimension reduced through the one or more feature extraction models. For example, if the reduced dimension is two-dimensional, one partition may have a grid shape, and if the reduced dimension is three-dimensional, one partition may have a cube shape.

The controller 140 applies the user's personal health data to the generated one or more feature extraction models and searches for public health data that is similar to the personal health data from the partitioned public health data using the personal health data of a dimension reduced by the feature extraction model.

In addition, the controller 140 may search for a partition matching the partition for public health data stored in the database 200 in advance by using the reduced-dimensional personal health data in order to search for similar cases.

In addition, the controller 140 may perform a 1:1 similarity calculation on the public health data belonging to the partition if there is a matching partition based on a result of the search. As a result of the similarity calculation, one or more higher-level public health data showing high similarity with the personal health data may be selected and the selected public health data may be outputted to the user.

Moreover, in order to calculate the 1:1 similarity, the high-speed similar case search device 100 may calculate the similarity using public health data and the original when the user's personal health data is inputted to the high-speed similar case search device 100, and the similarity calculation may use the Euclidean distance. However, the inventive concept may employ various similarity calculation methods including the Euclidian distance, the Manhattan distance, or the Hamming distance, and there is no limitation thereto.

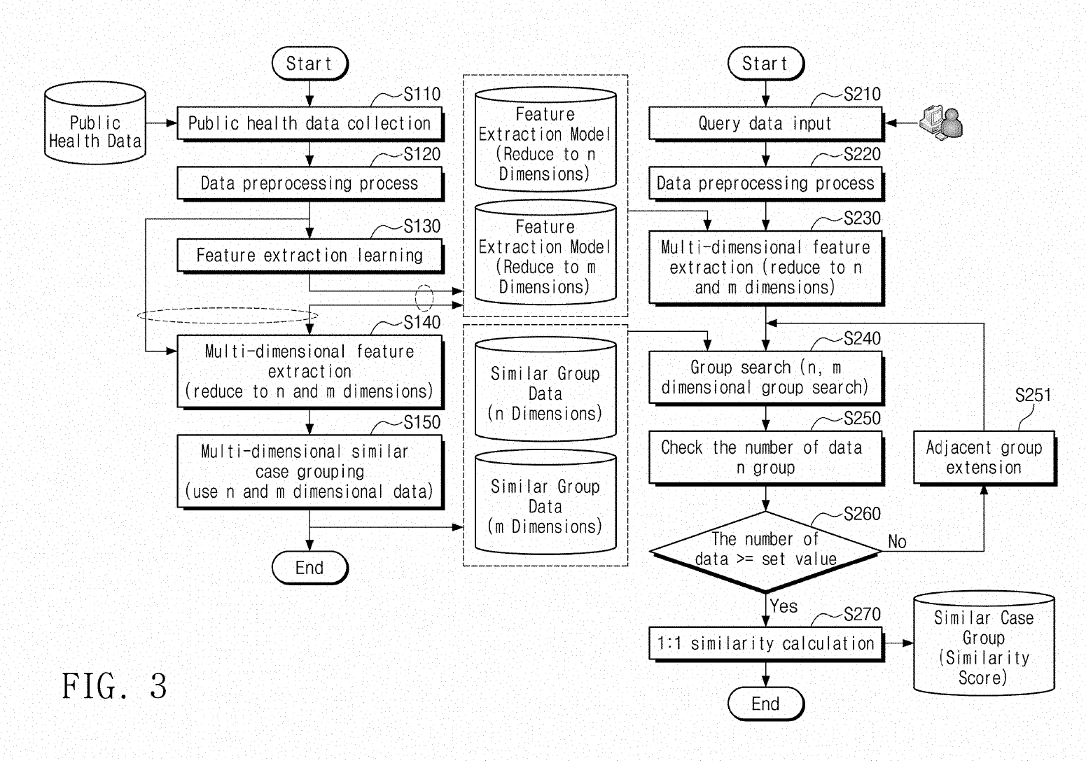

FIG. 3 is a workflow illustrating a method for searching for similar cases at high speed using personal health data of a user according to an embodiment.

As shown in FIG. 3, the high-speed similar case search device 100 may receive public health data for a high-speed similar case search (S110). The high-speed similar case search device 100 according to an embodiment may periodically collect public health data using a communication circuit. The high-speed similar case search device 100 according to an embodiment may receive public health data from a government agency or a user.

Next, the high-speed similar case search device 100 may perform a preprocessing process of converting health values for each health feature included in the input public health data into values between 0 and 1 (S120).

Next, the high-speed similar case search device 100 may generate one or more feature extraction models through multi-dimensional feature extraction learning on the public health data inputted to utilize the public health data as the target of the similar case search, and store them in the database 200 (S130). For example, the high-speed similar case search device 100 may generate one or more feature extraction models through machine learning on the inputted public health data. The high-speed similar case search device 100 may generate one or more feature extraction models by learning the public health data inputted through a machine learning technique for extracting features from specific data, such as PCA techniques, deep network learning techniques, SVD techniques, and so on.

Next, the high-speed similar case search device 100 may reduce the dimension of public health data by applying a plurality of preprocessed public health data to one or more feature extraction models (S140). For example, after loading one or more feature extraction models stored in the database 200 from the database 200 and then, applying the plurality of preprocessed public health data to the loaded one or more feature extraction models, the high-speed similar case search device 100 may reduce the dimension of the public health data by extracting features of the public health data for each feature extraction model.

Next, the high-speed similar case search device 100 may perform partitioning on the public health data of the reduced dimensions (S150). For example, the high-speed similar case search device 100 performs partitioning to group and store the public health data of the reduced dimensions for each feature extraction model by each partition, thereby establishing the database 200 for similar case search.

In addition, after the database 200 for similar case search is established, when the query data of the user is inputted to the high-speed similar case search device 100 (S210), the high-speed similar case search device 100 may perform a preprocessing process to convert the health values of each health feature included in the user's query data to values between 0 and 1. Thus, the health value may be converted into a state applicable to one or more feature extraction models stored in the database 200 (S220).

Moreover, the query data may include the entire personal health data of the user's multi-dimensional time series, or may include a portion of the personal health data.

Also, the query data may be inputted through a user interface provided by the high-speed similar case search device 100 or a user interface provided by a health check service system interlocked with the high-speed similar case search device 100.

Next, the high-speed similar case search device 100 may reduce the dimension of the query data of the user processed by the preprocessing process (S230). For example, the high-speed similar case search device 100 extracts features by applying the preprocessed query data of the user to the stored one or more feature extraction models, and then reduces the dimensions of the query data for each feature extraction model to output the query data of the reduced dimensions.

Next, the high-speed similar case search device 100 searches a partition stored in the database 200 and searches for a partition matching the converted query data (S240). The search may be performed by a partition unit, and a plurality of public health data mapped to the partition may be extracted by searching for a partition matching the partition of the query data.

Next, the high-speed similar case search device 100 determines the number of extracted public health data (i.e., the number of public health data belonging to the partition) (S250), and If the number of determined public health data is smaller than the set value (S260), the partition to be searched may be expanded to an adjacent partition (S251). Thus, a sample of public health data for the similarity computation may be extended.

The high-speed similar case search device 100 may repeatedly performs operations S240 to S260 through the expansion, and if the number of public health data for calculating the similarity is equal to or greater than the set value, extract the plurality of public health data from the database 200. The high-speed similar case search device 100 may perform the similarity calculation by comparing the extracted public health data with the query data 1:1 (S270).

Also, the high-speed similar case search device 100 may generate a similar case group for the corresponding query data by selecting a plurality of public health data having a high similarity score according to the performed similarity calculation. Also, the high-speed similar case search device 100 may store the generated similar case group and output the stored similar case group to the user.

Meanwhile, the public health data and the query data of the user used for calculating the similarity may refer to the original public health data and query data originally inputted into the database, instead of the public health data and the query data reduced to multi-dimensions for the similar case search.

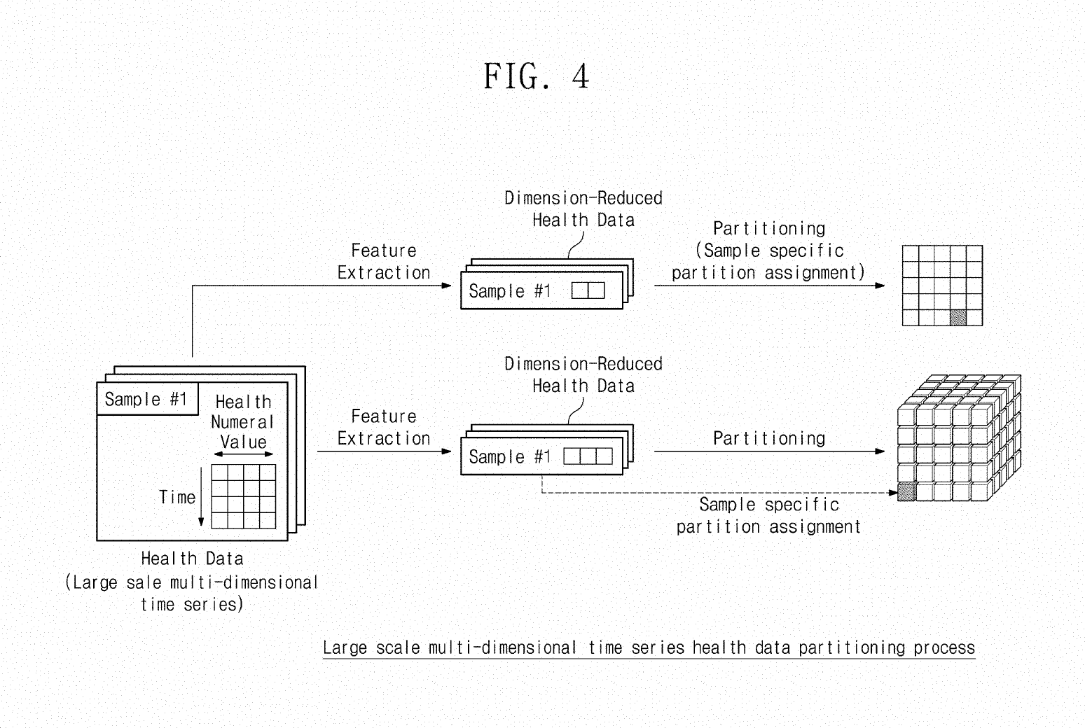

FIG. 4 is a diagram illustrating a process of partitioning large scale multi-dimensional time series health data according to an embodiment of the inventive concept.

As shown in FIG. 4, the partitioning of the multi-dimensional time series health data includes extracting features by applying a plurality of multi-dimensional time series health data to one or more feature extraction models, and reducing the plurality of multi-dimensional time series health data to multi-dimensions.

That is, the high-speed similar case search device 100 may reduce the dimensions (e.g., N-dimensions, N>3) of the inputted public health data or the original of the user's personal health data to dimensions (e.g., two-dimensions, three-dimensions, etc.).

The dimension reduction may be performed by each of the feature extraction models, and the feature extraction model may be designed to reduce the large scale multi-dimensional time series health data to two-dimensions, three-dimensions, or larger dimensions. For example, a feature extraction model may be obtained by mechanically learning inputted public health data.

Next, the high-speed similar case search device 100 performs partitioning according to the reduced dimension of the large-scale multi-dimensional time series health data, and assigns the health data mapped to each dimension as the partition for each section.

The partition is obtained by partitioning the space of each dimension based on an arbitrary range, as will be described below, and according to the partitioning result, 0 health data may belong to a specific partition. That is, each partition may be mapped to zero or more public health data.

Also, the partitions may be grouped according to a similar pattern (i.e., a pattern of the feature or health numerical value) between the public health data, and the public health data belonging to the partition may have similar features.

For example, multi-dimensional public health data may be reduced to two-dimensional or three-dimensional data through the high-speed similar case search device 100, and when the public health data is mapped onto the two-dimensional graph by treating each of the two-dimensional components (i.e., the above-mentioned features) as values of the x-axis and the y-axis, the health data may appear in the form of dots on the two-dimensional graph.

Moreover, each partition has a range of x values and a range of y values on the two-dimensional graph (i.e., two-dimensional space). The high-speed similar case search device 100 may store the x and y values for each partition in advance, and store them to quickly search for a similar case group through a simple range search and map new public health data to a corresponding partition.

For example, under the assumption that the range of x values and the range of y values for a particular partition are 0.1<x<0.2 and 0.1<y<0.2, when the health data having the values of <0.15, 0.15> in two dimensions are inputted through the high-speed similar case search device 100, the inputted health data may be matched to the specific partition simply searching for a range.

Also, when the health data is converted into three-dimensional data through the high-speed similar case search device 100, it may be partitioned into cubes and mapped to a three-dimensional graph (i.e., a three-dimensional space) through the high-speed similar case search device 100.

However, although FIG. 4 illustrates that the multi-dimensional time series health data is reduced to two-dimensional and three-dimensional multi-dimensions and partitioned, it is apparent that various types of partitioning may be performed depending on the reduction to two dimensions and three dimensions in addition to a larger dimension than that.

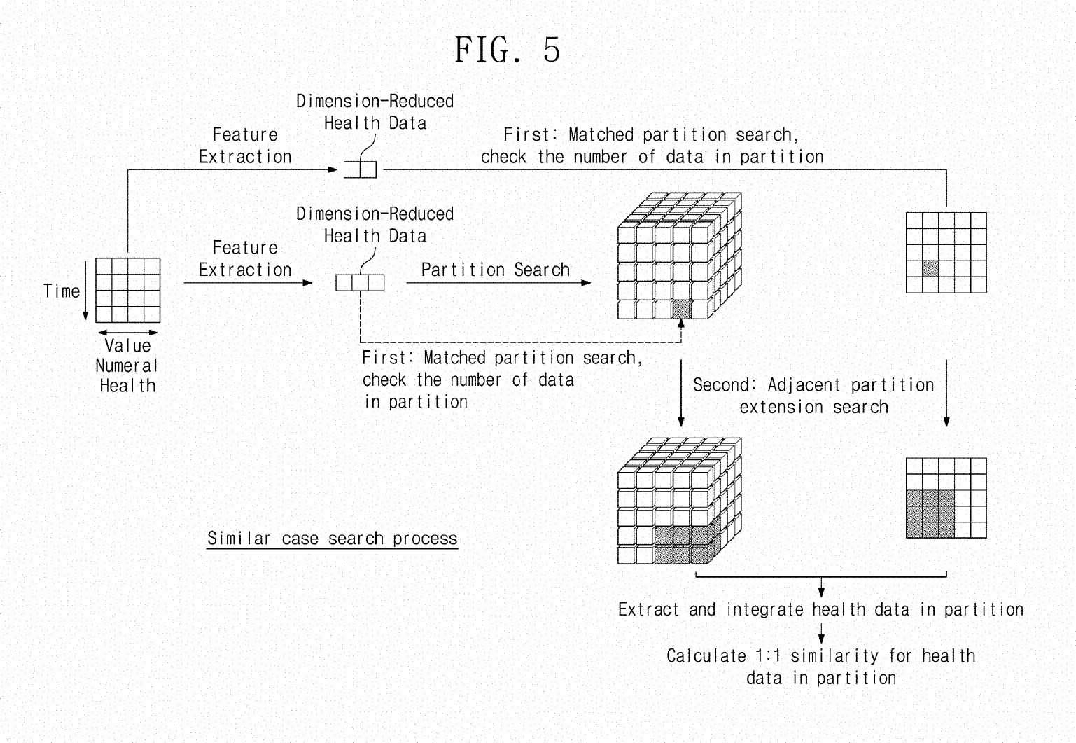

FIG. 5 is a diagram illustrating a process of searching for similar cases through multi-dimension reduction according to an embodiment.

As shown in FIG. 5, when a user's personal health data (i.e., query data) is inputted, the high-speed similar case search device 100 applies a plurality of feature extraction models to the personal health data to extract features, thereby performing multi-dimension reduction.

Next, the high-speed similar case search device 100 may search for a specific partition through the range search so as to search for similar cases similar to the personal health data of the user based on the personal health data of the reduced dimensions.

Next, the high-speed similar case search device 100 checks the number of a plurality of public health data grouped into a similar group in the found partition, and determines whether the checked number is equal to or greater than a predetermined number (for example, a threshold value).

If the checked number is less than the predetermined number based on the determination result, the extension search to the adjacent partition is repeatedly performed until the plurality of public health data becomes the predetermined number or more, so that a plurality of public health data may be extracted and integrated.

Since the user's personal health data may be similar to similar cases grouped in another partition adjacent to a corresponding partition in addition to similar cases in an initially found partition, the high-speed similar case search device 100 may also extract public health data in the adjacent partition and perform similarity calculation.

Accordingly, the high-speed similar case search device 100 may finely divide the range of partitions that partition the dimension space, and if the user's personal health data matches a particular partition, select public health data in a partition adjacent thereto in addition to a corresponding partition and calculate the similarity.

Next, the high-speed similar case search device 100 may perform similarity calculation by comparing the integrated public health data with the personal health data 1:1, and select public health data having a high similarity score to output the selected health data to a user.

Accordingly, the high-speed similar case search device 100 according to an embodiment reduces the n-dimensional health data into k-dimensional and l-dimensional multi-dimension health data to extract only the feature portion of the health data, thereby reducing the number of constraints when searching for similar cases. Also, the high-speed similar case search device 100 may significantly improve the similar case search speed and search for similar cases with high accuracy by partitioning the health data according to each dimension reduced to multi-dimensions.

As described above, the high-speed similar case search method for multidimensional health data and the device thereof allow searching for health data similar to a health state of a user based on the user's personal health data, thereby reducing the computational complexity of a similarity between the user's personal health data and the public health data and significantly reducing the time spent searching for similar cases.

In relation to a high-speed similar case search method and a device thereof through the large-scale multi-dimensional time series reduction to multi-dimensions, the dimensions of health data, which is big data, are reduced to multi-dimensions by applying machine learning techniques for feature extraction, so that the computational complexity of found similar cases of users is drastically reduced, and as a result, there is an effect that a case similar to a user may be searched at a high speed close to real-time.

Further, by applying a partitioning technique for grouping health data having similar characteristics into a plurality of similar groups, when the user's personal health data is inputted, it is determined which partition the personal health data belongs to, without performing the similarity calculation for all health data, so that the similarity calculation may be performed only for the similar group of the specific partition and as a result, it is possible to drastically reduce the time required to search for a case similar to the user's health condition.

Although the exemplary embodiments of the inventive concept have been described, it is understood that the inventive concept should not be limited to these exemplary embodiments but various changes and modifications can be made by one ordinary skilled in the art within the spirit and scope of the inventive concept as hereinafter claimed.

What is claimed is:

1. A method performed by a device including one or more processors for similar case search on multi-dimensional health data, the method comprising:
preprocessing health data inputted through an interface circuit;
performing a multi-dimensional feature extraction learning based on machine learning on the preprocessed health data; and
generating one or more feature extraction models for dimension reduction based on the multi-dimensional feature extraction learning.

2. The method of claim 1, further comprising:
reducing a dimension for a feature of health data by applying the preprocessed health data to the generated one or more feature extraction models;
extracting the feature of the reduced dimension; and
grouping the health data of the reduced dimension by each partition based on the extracted feature.

3. The method of claim 2, further comprising:
when personal health data of a user for a similar case search is inputted as query data through the interface circuit, preprocessing the query data;
reducing the dimension of the feature for the personal health data of the user by applying the preprocessed query data to the generated one or more feature extraction models; and
extracting the query data of the reduced dimension.

4. The method of claim 3, further comprising:
matching the query data of the reduced dimension to health data of a grouped partition;
calculating a similarity between the health data of the matched partition and the query data; and
outputting health data having the similarity that is greater than or equal to a set value.

5. The method of claim 4, wherein the calculating of the similarity comprises:
when the number of the health data of the matched partition is less than a critical value, matching health data of a partition adjacent to the matched partition to the query data of the reduced dimension; and
calculating the similarity between the health data of the adjacent partition and the query data.

6. The method of claim 1, wherein the one or more feature extraction models are generated by applying at least one of a Principal Component Analysis (PCA) technique, a Deep Network Learning technique, and a Singular Value Decomposition (SVD) technique.

7. A device configured to provide a similar case search on multi-dimensional health data, the device comprising:
an input/output interface configured to receive health data; and
a controller configured to preprocess the received health data and perform a multi-dimensional feature extraction learning based on machine learning on the preprocessed health data to generate one or more feature extraction models for dimension reduction.

8. The device of claim 7, wherein the controller is configured to reduce a dimension for a feature of health data by applying the preprocessed health data to the generated one or more feature extraction models,
extract the feature of the reduced dimension, and
group the health data of the reduced dimension by each partition based on the extracted feature.

9. The device of claim 8, wherein when personal health data of a user for a similar case search is inputted as query data through the interface circuit, the controller is further configured to preprocess the query data,
reduce the dimension of the feature for the personal health data of the user by applying the preprocessed query data to the generated one or more feature extraction models, and
extract the query data of the reduced dimension.

10. The device of claim 9, wherein the controller is further configured to match the query data of the reduced dimension to health data of a grouped partition,
calculate a similarity between the health data of the matched partition and the query data; and
output health data having the similarity that is greater than or equal to a set value.

11. The device of claim 10, wherein in order to output the health data having the similarity that is greater than or equal to the set value, the controller is further configured to, when the number of the health data of the matched partition is less than a critical value, match health data of a partition adjacent to the matched partition to the query data of the reduced dimension, and calculate the similarity between the health data of the adjacent partition and the query data.

12. The device of claim 7, wherein the one or more feature extraction models are generated by applying at least one of a Principal Component Analysis (PCA) technique, a Deep Network Learning technique, and a Singular Value Decomposition (SVD) technique.
